(19) United States
(12) Patent Application Publication
(10) Pub. No.: US 2012/0010643 A1
(43) Pub. Date: Jan. 12, 2012

(54) PUNCTURE NEEDLE FOR ORTHOPEDIC OPERATION

(76) Inventor: Weixing Shao, Shandong (CN)

(21) Appl. No.: 13/257,600

(22) PCT Filed: Jun. 29, 2009
(86) PCT No.: PCT/CN2009/000723

§ 371 Date: Sep. 20, 2011

(30) Foreign Application Priority Data
Apr. 3, 2009 (CN) 200920020328.4

(51) Int. Cl. A61B 17/34

(52) U.S. Cl. 606/185

(57) ABSTRACT

The present invention offers a program of a medical surgery device, especially a puncture needle for bone surgery. The program includes a needle tube and a needle tube seat for fastening the needle tube, and a needle core with cutting edge inserting into the needle tube and a needle core seat for fastening the needle core, and a hollow bolt through needle core is set on the needle tube seat, and in the program, a nut matching with the hollow bolt is fastened on the said needle core seat, the nut and the needle core are coaxial. The said hollow bolt has a Luer conical fitting. The said bolt and the needle core and the needle core seat are one structure.

### BACKGROUND OF THE INVENTION

The present invention relates to a kind of medical device, especially a puncture needle for bone surgery.

In existing technologies, the widely-known technology of puncture needle is in use now, the blade of the existing puncture needle is extruded out of the needle tube, it would get a big resistance when plugged into a bone, and made the needle core shrinked into the interior of the needle tube to bring inconvenience for surgery; and it's hard for needle core to leave after punctureing finished because the big density bone clamped it, and when serious, the needle core even can be separated from needle core seat, that causes a unnecessary trouble for surgery, and can not make it proceed smoothly. Those are the shortcomings of the existing technologies.

### BRIEF SUMMARY OF THE INVENTION

The purpose of the present invention is to offer a technical program of a puncture needle for bone surgery aiming at the shortcomings of existing technologies. In the program, a nut matching with a hollow bolt is fastened on the needle core seat, the nut can be screwed into the hollow bolt with needle core to make the needle core seat and needle tube seat fixed, which is conductive for insertion of needle and withdraw of needle core.

The program is achieved through the following technical measures: the puncture needle includes a needle tube and a needle tube seat for fastening the needle tube, a needle core with blade inserting into the needle tube and a needle core seat for fastening the needle core, and also a hollow bolt through the needle core on the needle tube seat, the features of the program are that a nut matching with a hollow bolt is fastened on the said needle core seat, the nut and the said needle core are coaxial. The specific features of the program also include that the said hollow bolt has a Luer conical fitting. The said nut and needle core and needle core seat are one structure.

The beneficial effects of the program can be recognized by the above narration, in the program, a nut matching with a hollow bolt is fastened on the said needle core seat, the nut and the said needle core are coaxial. In this structure, the nut on the needle core seat can be spun into the hollow bolt to make the needle core seat and needle tube seat fasten, when inserting into bone, the needle core seat and needle tube seat can not be separated because of connection of nut and hollow bolt, it is conducive for the insertion of puncture needle; and it is easy for exiting of the needle core, as long as rotating the needle core seat to separate the nut and the hollow bolt connected with thread, the operation is very convenient to ensure it completed smoothly. Therefore, compared to the existing technologies, the present invention has substantive features and progress, it is also obvious for its beneficial effects of implementation.

### BRIEF DESCRIPTION OF THE DRAWINGS

FIG. 1 is structure view of embodiment of the invention.

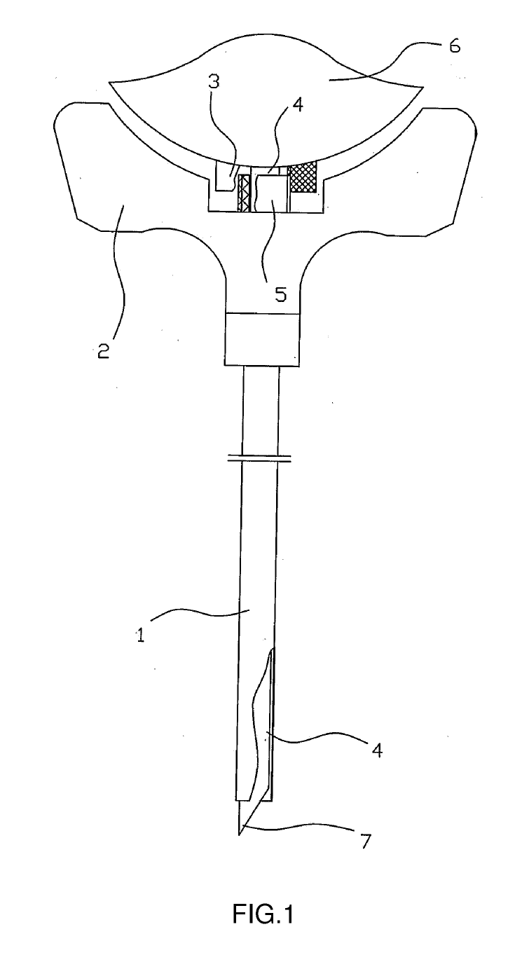

FIG. 2 is the left view of the FIG .1.

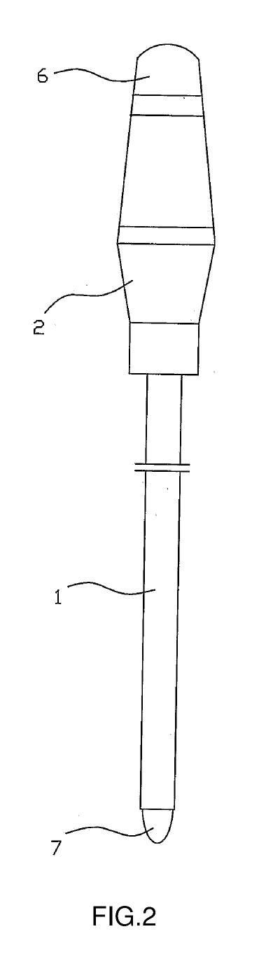

Where, 1 is needle tube, 2 is needle tube seat, 3 is nut, 4 is needle core, 5 is hollow bolt, 6 is needle core seat, 7 is cutting edge.

### DETAILED DESCRIPTION OF THE INVENTION

In order to clearly explain the technical features of the program, the following will describe the program by a embodiment and its drawings.

It can be seen from the drawings, the puncture needle for bone surgery of the program includes a needle tube 1 and a needle tube seat 2 for fastening the needle tube 1, and a needle core 4 with cutting edge inserting into the needle tube 1 and a needle core seat 6 for fastening the needle core 4, and a hollow bolt 5 through needle core 4 on the needle tube seat 2, the hollow bolt 5 has a Luer conical fitting. In the program, a nut matching with the hollow bolt 5 is fastened on the said needle core seat 6, the nut 3 and the needle core 4 and needle core seat 6 are one structure by injection molding, adhering or assembling. In addition, the said nut 3 and the said needle core 4 are coaxial.

What is claimed is:

1. A puncture needle for bone surgery includes a needle tube and needle tube seat for fastening the needle tube, and a needle core with cutting edge inserting into the needle tube and a needle core seat for fastening the needle core, and a hollow bolt through needle core on the needle tube seat, wherein a nut matching with the hollow bolt is fastened on the said needle core seat, the nut and the needle core are coaxial.

2. The puncture needle for bone surgery of claim 1, wherein the said hollow bolt has a Luer conical fitting.

3. The puncture needle for surgery of claim 1 or 2, wherein the said nut and the needle core and the needle core sear are one structure.
